(19) United States
(12) Patent Application Publication
(10) Pub. No.: US 2011/0301117 A1
(43) Pub. Date: Dec. 8, 2011

(54) CRYOGENIC SKIN CREAM AND METHOD

(76) Inventor: Tim Schmidt, Miami Beach, FL (US)

(21) Appl. No.: 12/793,530

(22) Filed: Jun. 3, 2010

(51) Int. Cl. A61K 31/728; A61Q 19/00; A61K 31/01

(52) U.S. Cl. 514/54; 514/762

(57) ABSTRACT

A skin cream and skin cream packaging arrangement is disclosed. The arrangement includes, in combination: a skin cream intended to be applied on human skin and found to be most effective when within a determinable temperature range, and a container for containing the skin cream. The container may have a temperature indicator thereon configured to provide a visible indicator when the container is within the determinable temperature range.

### BACKGROUND

This application relates generally to skin creams. More specifically, this application relates to a skin cream having effectiveness advantages in a predetermined temperature range, and which is contained in a container providing an indication as to when and if the skin cream is within the predetermined temperature range.

### SUMMARY

Currently skin creams formulations and methods of using them rely solely on the effectiveness provided by their constituent ingredients, and there relative quantities thereof, on the quantity used, and on frequency of application. Oftentimes the most effective ingredients in skin cream formulations are the most costly.

Therefore, there is a significant need to increase the effectiveness of skin creams without increasing the quantities used, the frequency of application, or the amount of costly ingredients. There is also a significant need to provide a means to help the user use the skin cream under conditions of peak effectiveness. The applicant of the present disclosure has discovered a skin cream formulation which when kept within a predetermined temperature range has an increased effectiveness.

This application discloses a skin cream and container arrangement that is economical to produce, of simple formulation and construction, and capable of mass production, but also capable of providing a user a skin cream having superior effectiveness.

In particular, this application discloses a skin cream and skin cream packaging arrangement, including, in combination: a skin cream intended to be applied on human skin and found to be most effective when within a determinable temperature range, and a container for containing the skin cream. The container may have a temperature indicator thereon configured to provide a visible indicator when the container is within the determinable temperature range.

This application also discloses a product including a cream intended to be applied on human skin and formulated to be most effective when the skin cream is within a predetermined temperature range. The product may also include a container for containing the cream having a temperature indicator thereon configured to provide a human perceptible indication when the container is within the predetermined temperature range.

Various operations in the current disclosure may be described as multiple discrete operations in turn, in a manner that may be helpful in understanding embodiments of the present invention. However, the order of description should not be necessarily be construed to imply that these operations are order dependent.

Finally, this application discloses a method of using a skin cream. The method may include cooling a skin cream which is formulated to be most effective when within a predetermined temperature range to within the predetermined temperature range. The method may also include applying the skin cream to the skin of a user when the skin cream is within the predetermined temperature range.

### BRIEF DESCRIPTION OF THE DRAWINGS

The drawings, when considered in connection with the following description, are presented for the purpose of facilitating an understanding of the subject matter sought to be protected.

FIG. 1 is a front view illustrating a container containing a skin cream in accordance with various embodiments;

FIG. 2 is a flow diagram illustrating a first method in accordance with various embodiments; and

FIG. 3 is a flow diagram illustrating a second method which may be a variation of the method illustrated in FIG. 2, in accordance with various embodiments.

### DETAILED DESCRIPTION

While the present skin cream and container arrangement is described with reference to several illustrative embodiments described herein, it should be clear that the present invention should not be limited to such embodiments. Therefore, the description of the embodiments provided herein is illustrative of the present invention and should not limit the scope of the invention as claimed. In addition, while the following description references drawings showing particular configurations and proportions, it will be appreciated that the invention may be configured to have other configurations and proportions.

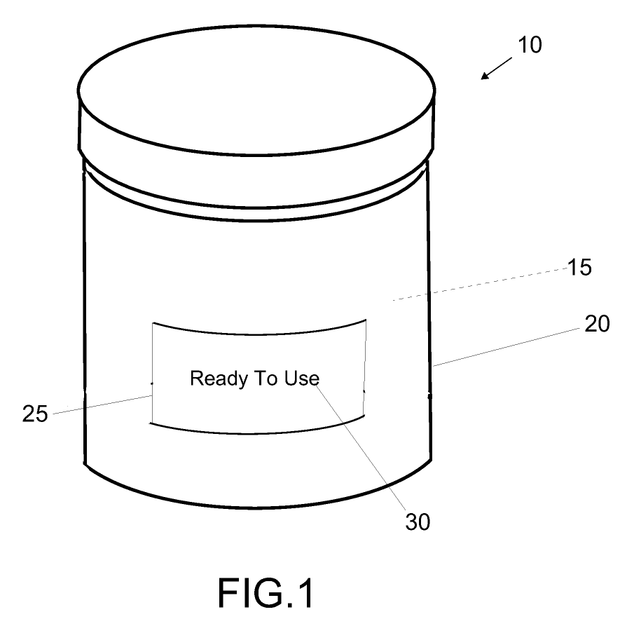

Referring now to FIG. 1, a first embodiment of a skin cream, and skin cream packaging arrangement 10 is disclosed. The arrangement 10 may include, in combination, a skin cream 15, and a container 20 for containing the skin cream 15. The skin cream 15 may be intended to be applied on human skin and may be found to be most effective when it is within a determinable temperature range. The container may have a temperature indicator 25 thereon configured to provide a visible indication to communicate to a user, or potential user, when the container 20, and therefore the skin cream, is within the determinable temperature range.

FIG. 1 also illustrates a product 10 including a cream 15 that may be intended to be applied on human skin, and may be formulated to be most effective when within a predetermined temperature range. The product 10 may also include a container 20 for containing the cream 15. The container 20 may have temperature indicator 25 thereon configured to provide a human perceptible indication 30 when the container 20 is within the predetermined temperature range.

Human perceptible indication 30 may be provided various ways, including visually, or audibly, or other ways. In some cases the human perceptible indication may be a visible indicator that may be, for example, a change of color. In some cases the human perceptible indication 30 may be an appearance change, for example, an appearance of one or more shapes, or depiction of an object, or an appearance of one or more letters that may provide a message, as shown in the example illustrated in FIG. 1, indicting to the user that the skin cream is within the predetermined temperature range and ready for use.

In some cases the temperature indicator 25 may be included in a label affixed to the container. The term “affixed to” may be understood to be, for example, glued, or held on with an electrostatic charge, or otherwise held in thermal transfer contact with the container.

In some embodiments the determinable temperature range, or predetermined temperature range, may be between 36 and 45 degrees. In some embodiments the determinable temperature range, or predetermined temperature range, may be between 39 and 41 degrees.

In some embodiments the skin cream may include squalane, and hyaluronic acid. squalane, and/or hyaluronic acid may be active ingredients. In some embodiments the skin cream may include: purified water (aqua), squalane, hyaluronic acid, polyacrylate-13, polyisobutene, polysorbate 20, xylitylglucoside, anhydroxylitol, xylitol, phenoxyethanol, and ethylhexylglycerin. In some embodiments the skin cream may include, in descending relative quantities, purified water (aqua), squalane, hyaluronic acid, polyacrylate-13, polyisobutene, polysorbate 20, xylitylglucoside, anhydroxylitol, xylitol, phenoxyethanol, and ethylhexylglycerin.

In some embodiments one or more of the other listed ingredients may be replaced by other appropriate substitutes without departing from the scope of the present disclosure. For example, in some cases, one or more other ingredients having appropriate plasticity characteristics may be used as a substitute for polyacrylate-13. In some cases, one or more other appropriate viscoelastic ingredients may be used as a substitute for polyisobutylene. In some cases, one or more other appropriate ingredients that may have cleaning, wetting, or emulsifying properties may be used as a substitute for the polysorbate 20. In some cases, one or more other appropriate ingredients that may have properties to aid in dermal moisture retention may be used as a substitute for the xylitylglucoside, and/or anhydroxylitol, and/or xylitol. In some cases, one or more other appropriate ingredients that may have preservative properties may be used as a substitute for the phenoxyethanol and/or the ethylhexylglycerin.

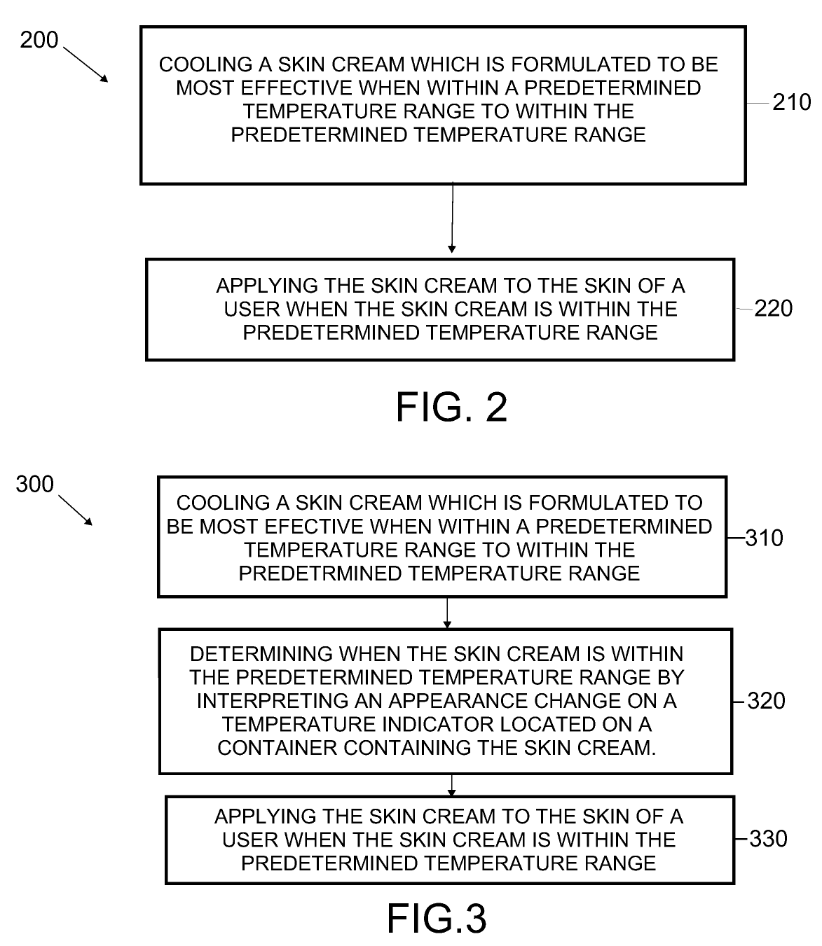

FIG. 2 is a flow diagram illustrating a method 200 in accordance with various embodiments. The method 200 may include, at 210, cooling a skin cream which is formulated to be most effective when within a predetermined temperature range to within the predetermined temperature range. The method 200 may include, at 220, applying the skin cream to the skin of a user when the skin cream is within the predetermined temperature range.

FIG. 3 is a flow diagram illustrating another method 300 in accordance with various embodiments. The method 300 may be a variation of the method 200 illustrated in FIG. 2 that may include, at 310, cooling a skin cream which is formulated to be most effective when within a predetermined temperature range to within the predetermined temperature range. The method 300 may include the intermediate step, at 320, determining when the skin cream is within the predetermined temperature range by interpreting an appearance change on a temperature indicator located on a container containing the skin cream. The interpreting may be, for example, recognizing that the appearance change indicates the skin cream is within the predetermined temperature range and ready to use. The method 300 may include a final step, at 330, applying the skin cream to the skin of a user when the skin cream is within the predetermined temperature range.

In some cases, the method may include formulating the skin cream to include squalane, and hyaluronic acid. In some cases, the method may also include formulating the skin cream to also include polyacrylate-13, polyisobutene, polysorbate 20, xylitylglucoside, anhydroxylitol, xylitol, phenoxyethanol, and ethylhexylglycerin.

In some cases, the predetermined temperature range may be between 36 and 45 degrees. In some cases, the predetermined temperature range may be between 39 and 41 degrees.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. A skin cream and skin cream packaging arrangement, comprising, in combination:
a skin cream intended to be applied on human skin and found to be most effective when within a determinable temperature range; and
a container for containing the skin cream, the container having a temperature indicator thereon configured to provide a visible indicator when the container is within the determinable temperature range.

2. The skin cream and skin cream packaging arrangement of claim 1, wherein the visible indicator is a change of color.

3. The skin cream and skin cream packaging arrangement of claim 1, wherein the visible indicator is an appearance of one or more shapes, or one or more letters on the temperature indicator.

4. The skin cream and skin cream packaging arrangement of claim 1, wherein the temperature indicator is included in a label affixed to the container.

5. The skin cream and skin cream packaging arrangement of claim 1, wherein the determinable temperature range is between 36 and 45 degrees.

6. The skin cream and skin cream packaging arrangement of claim 1, wherein the determinable temperature range is between 39 and 41 degrees.

7. The skin cream and skin cream packaging arrangement of claim 1, wherein the skin cream includes squalane, and hyaluronic acid.

8. The skin cream and skin cream packaging arrangement of claim 1, wherein the skin cream includes: purified water (aqua), squalane, hyaluronic acid, polyacrylate-13, polyisobutene, polysorbate 20, xylitylglucoside, anhydroxylitol, xylitol, phenoxyethanol, and ethylhexylglycerin.

9. The skin cream and skin cream packaging arrangement of claim 1, wherein the skin cream includes in descending relative quantities: purified water (aqua), squalane, hyaluronic acid, polyacrylate-13, polyisobutene, polysorbate 20, xylitylglucoside, anhydroxylitol, xylitol, phenoxyethanol, and ethylhexylglycerin.

10. A product comprising:
a cream intended to be applied on human skin and formulated to be most effective when within a predetermined temperature range; and
a container for containing the cream having a temperature indicator thereon configured to provide a human perceptible indication when the container is within the predetermined temperature range.

11. The product of claim 1, wherein the cream has active ingredients including squalane, and hyaluronic acid.

12. The product of claim 1, wherein the cream includes squalane, and hyaluronic acid.

13. The product of claim 1, wherein the predetermined temperature range is between 36 and 45 degrees.

14. The product of claim 1, wherein the predetermined temperature range is between 39 and 41 degrees.

15. A method comprising:
cooling a skin cream which is formulated to be most effective when within a predetermined temperature range to within the predetermined temperature range; and
applying the skin cream to the skin of a user when the skin cream is within the predetermined temperature range.

16. The method of claim 15, further comprising determining when the skin cream is within the predetermined temperature range by interpreting an appearance change on a temperature indicator located on a container containing the skin cream.

17. The method of claim 15, further comprising formulating the skin cream to include Squalane, and Hyaluronic Acid.

18. The method of claim 15, further comprising formulating the skin cream to also include polyacrylate-13, polyisobutene, polysorbate 20, xylitylglucoside, anhydroxylitol, xylitol, phenoxyethanol, and ethylhexylglycerin.

19. The method of claim 15, wherein the predetermined temperature range is between 36 and 45 degrees.

20. The method of claim 15, wherein the predetermined temperature range is between 39 and 41 degrees.
